(19) United States
(12) Patent Application Publication
(10) Pub. No.: US 2026/0207530 A1
(43) Pub. Date: Jul. 23, 2026

(54) USE FOR AMELIORATION OF INTESTINAL DISEASES USING ALLULOSE

(71) Applicant: SAMYANG CORPORATION, Seoul (KR)

(72) Inventors: Soonok SA, Yongin-si (KR); Goeun KIM, Seongnam-si (KR); Kyunghun RYU, Seongnam-si (KR); Seyoung PARK, Suwon-si (KR); Jiwon PARK, Seongnam-si (KR); Jungsook HAN, Anyang-si (KR); Hwanju HWANG, Seoul (KR)

(21) Appl. No.: 19/139,469

(22) PCT Filed: Dec. 21, 2023
(86) PCT No.: PCT/KR2023/021223

§ 371 Date: Jun. 16, 2025

(30) Foreign Application Priority Data
Dec. 30, 2022 (KR) 10-2022-0191203

(51) Int. Cl. A61K 31/121; A61K 9/00; A61P 1/04

(52) U.S. Cl. CPC A61K 31/121; A61K 9/0053; A61P 1/04

(57) ABSTRACT

The present disclosure relates to a composition containing allulose as an active ingredient, and to an use of the composition for preventing, improving, or treating intestinal diseases, such as leaky gut, and/or symptoms of leaky gut.

### TECHNICAL FIELD

The present invention relates to a composition for preventing, improving or treating an intestinal disease, comprising allulose as an active ingredient, and a composition for preventing, improving or treating an intestinal disease, such as leaky gut or a symptom of gut leakage using the composition.

### TECHNICAL FIELD

The functional intestinal barrier is essential for maintaining the homeostasis of the intestine and the whole body, separating the internal environment, which is strictly controlled, from the external environment, and simultaneously absorbing nutrients and body fluids while preventing the invasion of pathogens and other harmful molecules (e.g., pro-inflammatory bacterial products).

The intestinal epithelial layer is a major determinant of mucosal barrier function, and the tight junction complex seals the paracellular space between adjacent epithelial cells and plays a central role in the paracellular pathway. Impaired tight junction function is known to induce local and systemic inflammatory and immune responses, ultimately triggering and/or sustaining systemic inflammatory disorders. In other words, maintaining a homeostatic intestinal epithelial barrier is of utmost importance, and a reduction or disruption of barrier function is directly associated with increased paracellular and transcellular intestinal permeability, which is characterized by increased invasion of pathogens and the translocation of pro-inflammatory luminal factors into the systemic circulation, leading to subsequent systemic inflammation and the onset of numerous diseases. The so-called “leaky gut syndrome” is not only associated with various chronic inflammatory intestinal diseases and conditions, such as inflammatory bowel disease (IBD), irritable bowel syndrome (IBS), and celiac disease, but is also linked to a wide range of extraintestinal/systemic diseases and disorders. Increased intestinal permeability is associated with conditions of both intestinal and systemic disease, including inflammatory bowel disease and neurodegenerative diseases such as Parkinson's disease.

In recent years, there has been increasing interest in factors capable of enhancing the intestinal epithelial barrier through barrier-protective or restorative properties. A widely adopted therapeutic approach is the restoration of dysbiosis gut microbiota, wherein modulation of the symbiotic microbiome through bacteria-based therapy solves microbial imbalance, thereby alleviating inflammation and restoring healthy microbiota. Such bacteria-based therapies now include traditional probiotics, prebiotics, or a combination thereof. Prebiotics serve an indirect function in promoting enhanced barrier function, in consideration of their ability to stimulate the growth of beneficial symbiotic organisms.

Accordingly, there is an emerging need for substances that can directly function to prevent, alleviate, or treat homeostatic imbalance of functional intestinal barrier and the resulting leaky gut or leaky gut syndrome, with little concerns for side effects or safety.

### DISCLOSURE

### Technical Problem

An embodiment of the present invention relates to a composition for the prevention, improvement, or treatment of intestinal diseases, such as inflammatory bowel disease, leaky gut, and/or leaky gut syndrome, comprising allulose as an active ingredient.

An additional embodiment of the present invention relates to a composition for the prevention, improvement, or treatment of intestinal diseases, such as inflammatory bowel disease, gut leakage, and/or leaky gut syndrome, comprising allulose as an active ingredient, wherein the composition reduces the expression levels of factors involved in the Toll-like receptor 4 (TLR4) signaling pathway.

### Technical Solution

An embodiment of the present invention relates to a composition for the prevention, improvement, or treatment of intestinal diseases, such as inflammatory bowel disease, gut leakage, and/or leaky gut syndrome, comprising allulose as an active ingredient.

In a specific embodiment, the present invention relates to a composition for improving intestinal barrier cell permeability comprising allulose as an active ingredient, and more specifically, the composition can be prevent damage to intestinal barrier cell permeability or improve impaired intestinal barrier cell permeability, thereby preventing, improving, or treating leaky gut or leaky gut syndrome caused by an increased intestinal barrier cell permeability.

In the present specification, the terms “leaky gut” or “leaky gut syndrome (LGS)” collectively refer to a phenomenon in which the permeability of the intestinal membrane increases when a stimulus or damage is applied, allowing macromolecules to pass back and forth through the gaps between cells, and refers to a range of symptoms caused by the impaired function of the intestinal barrier, in which macromolecules in the bloodstream leak into the intestinal lumen or macromolecules in the lumen directly enter the bloodstream (a condition known as “leaky gut”).

The symptoms of leaky gut syndrome appear in various clinical conditions such as aging, allergies, multiple trauma, rheumatoid arthritis, inflammatory bowel disease, chronic fatigue syndrome, and irritable bowel syndrome. In addition, due to increased intestinal mucosal permeability or damage to the intestinal mucosa, pathogens, antigens, and putrefactive substances may enter the intestinal mucosa, which causes an inflammatory response, and endotoxins may enter the bloodstream, which causes bacterial translocation and intestinal endotoxemia, resulting in various inflammatory and immune responses.

In a specific embodiment, the gut leakage may result from increased intestinal mucosal permeability, damage to the intestinal mucosa, and/or intestinal inflammation. In addition, the gut leakage may be caused by decreased expression of TLR4, increased expression of prostaglandin E2 and COX-2, reduced intercellular adhesion strength between intestinal epithelial cells, or increased expression of ZO-1 and occludin.

Accordingly, in an embodiment, the prevention, improvement, or treatment of leaky gut syndrome may include preventing, suppressing, or treating a condition in which the intestinal mucosa permeability is increased due to loosening of the intercellular junctions between intestinal mucosal cells, thereby allowing the bidirectional passage of macromolecules.

In the present specification, the use of allulose as an active ingredient for the prevention, improvement, or treatment of intestinal diseases, such as inflammatory bowel disease, leaky gut, and/or leaky gut syndrome, may be attributable to at least one efficacies of allulose selected from the group consisting of an enhancing activity of intestinal mucosal immunity, an anti-inflammatory activity against intestinal inflammation, a protective activity of intestinal mucosa, an improving activity of intestinal permeability, and a scavenging activity of reactive oxygen species (ROS).

Specifically, the enhancing activity of intestinal mucosal immunity may be regulation of expression of factors involved in TLR4/NF-κB signaling and TLR4/MAPK signaling, or preferably at least a factor involved in TLR4/NF-κB signaling selected from the group consisting of p50, p65, TNF-60 , IL-6, IL-8, COX-2, TLR4, MyD88, IKKα, IKKβ, IκBα, IRAK4, and TRAF6; or at least a factor involved in MAPK signaling selected from the group consisting of p38, p-p38, JNK, p-JNK, c-Jun, p-c-Jun, c-Fos, and p-c-Fos,
the anti-inflammatory activity may be reduction or inhibition of expression of at least a factor selected from the group consisting of prostaglandin E2 as a pro-inflammatory mediator, and COX-2 as a key marker of inflammatory bowel disease,
the protective activity of intestinal mucosa may be reduction or inhibition of expression of at least one selected from the group consisting of MUC2, MUC5AC, and MUC5B as a mucin protecting the intestinal mucosa,
the improving activity of intestinal permeability may be improvement of increased intestinal permeability induced by barrier damage which is caused by inflammatory factors, enhancement of intercellular adhesion strength between intestinal epithelial cells, or increased expression of ZO-1 and Occludin as tight junction proteins, or
the ROS scavenging activity may be caused by increased expression of at least one selected from the group consisting of PI3K, AKT, Nrf2, and HO-1, as factors involved in the Nrf2/HO-1 signaling pathway activated by ROS.

In addition, in the present specification, allulose may be used as a TLR4 inhibitor or antagonist, and thus, may be provided for the prevention, improvement, or treatment of intestinal diseases, such as inflammatory bowel disease, intestinal leakage, and/or leaky gut syndrome, which are caused by TLR4-related signaling pathways.

Specifically, the allulose as a TLR4 inhibitor may regulating the expression of at least one selected from the group consisting of p50, p65, TNF-α, IL-6, IL-8, COX-2, TLR4, MyD88, IKKα, IKKβ, IκBα, IRAK4, and TRAF6 as factors involved in the TLR4/NF-κB signaling pathway.

In a specific embodiment of the present invention, allulose showed an anti-inflammatory effect by showing a decrease in the expression level of factors related to TLR4/NF-κB signaling as an inflammatory response-mediated signaling, in Caco-2 cells induced with inflammation by LPS, through RT-PCR and Western blot. An additional embodiment of the present invention relates to a composition comprising allulose as an active ingredient for reducing or improving intestinal permeability and for regulating inflammation caused by intestinal permeability. The present invention relates to the function of allulose in protecting or restoring the intestinal epithelial barrier. Accordingly, the present invention relates to a use of allulose in directly regulating tight junctions of intestinal epithelial cells, and more specifically, to the improvement or treatment of tight junctions and intestinal permeability associated with TLR4 signaling factors, or to the prevention or inhibition of increased tight junction disruption and increased intestinal permeability associated with TLR4 signaling factors.

The functionality of allulose according to the present invention is distinct from that of conventional prebiotics, which exert indirect effects on enhancing intestinal barrier function by promoting the growth of beneficial symbiotic microorganisms. In contrast, allulose is directly involved in the improvement, maintenance, and prevention of deterioration of the homeostatic intestinal epithelial barrier, etc.

The present invention exerts independently beneficial effects on the gut microbiota through direct interaction with intestinal epithelial and immune cells, and one of the functions is to promote the intestinal barrier function by directly regulating tight junctions through AMPK-, PKC-, MAPK-, and TLR-related pathways. The present invention aims to provide protective effects on tight junctions independently of, or separately from, the microbial community. Accordingly, the prevention, improvement, or treatment of leaky gut or leaky gut syndrome may be achieved not only directly through the prevention, improvement or treatment of leaky gut or leaky gut syndrome itself, but also optionally through the prebiotic activity of allulose, which modulates the growth of intestinal microbiota. Of course, the improvement of intestinal barrier permeability by allulose according to the present invention may result directly from the modulation of barrier permeability, but may also optionally include conventional prebiotic functionality.

The composition according to the present invention may be applied as a pharmaceutical composition or a food composition.

In the present specification, the efficacy of allulose in the prevention, improvement, or treatment of intestinal diseases was verified through specific experiments. That is, when an inflammatory response was induced by LPS treatment, the expression of TLR4 and NF-κB p65 genes increased. However, allulose treatment reduced the expression level of TLR4 activated by LPS, and consequently, the expression levels of NF-κB p65 and p50 were also decreased. These results confirmed the preventive anti-inflammatory effect of allulose through the suppression of TLR4/NF-KB signaling-related factors in intestinal epithelial cells in which inflammation was induced.

In addition, in HT-29 cells in which inflammation was induced by LPS, allulose reduced the expression level of TLR4 as an initiating signal mediating the inflammatory response. Consequently, the expression levels of activated MyD88, IRAK4, TRAF6, NF-κB signaling components (p50, p65, p-p65, IKKα, IKKβ, p-IKKα/β, IκBα, and p-IκBα), and MAPK signaling components (p38, p-p38, JNK, p-JNK, c-Jun, p-c-Jun, c-Fos, and p-c-Fos) were decreased, leading to a reduction in COX-2 expression. This confirmed the anti-inflammatory effect of allulose by showing a decrease in the expression levels of factors related to TLR4/NF-κB signaling and TLR4/MAPK signaling through RT-PCR and Western blot.

Additionally, in Caco-2 cells in which inflammation was induced by LPS, the expression levels of prostaglandin E2 as a pro-inflammatory mediator and COX-2 as a key marker of inflammatory bowel disease, were assessed using ELISA and RT-PCR. It was confirmed that the effect of regulating inflammation was observed by showing a decrease in the concentration of prostaglandin E2 and the expression level of COX-2.

In HT-29 cells with LPS-induced inflammation, the expression levels of mucins associated with intestinal barrier protection were assessed by RT-PCR. It was confirmed that allulose protected the intestinal tract by showing a decrease in the expression levels of MUC2, MUC5AC, and MUC5B.

The graph showing the values of electrical resistance (TEER) decreased by IFN-γ/TNF-α in polarized Caco-2 cells and the values of electrical resistance (TEER) increased by allulose is shown in FIG. 8. The values of electrical resistance (TEER) decreased by IFN-γ/TNF-α in polarized Caco-2 cells and the values of electrical resistance (TEER) increased by allulose were confirmed. The values of electrical resistance (TEER) increased by allulose show that allulose, as an active ingredient, improves the adhesive strength between intestinal epithelial cells.

By using FITC-dextran (4 kDa) in polarized Caco-2 cells, the fluorescence intensity increased by IFN-γ/TNF-α treatment was reduced by treatment with allulose, indicating that allulose improved paracellular permeability.

Additionally, using the DCFH-DA fluorescent probe in Caco-2 cells with LPS-induced inflammation, the accumulation of reactive oxygen species (ROS) was reduced by treatment with allulose, indicating that allulose contributed to the reduction of intestinal permeability.

In Caco-2 cells with LPS-induced inflammation, the reduced mRNA and protein expression levels of ZO-1 and occluding as the tight junction factors due to inflammation, were increased by treatment with allulose. These indicate that intestinal permeability, specifically, the tight junctions impaired by inflammation was improved by allulose treatment.

In the present specification, allulose is included as an active ingredient, in which allulose may be used alone or in a saccharide composition comprising allulose, or may be used in liquid or powder form, and the powder may be amorphous or crystalline. Allulose may be obtained from commercially available products or prepared using a predetermined raw material.

The allulose or saccharide composition containing allulose that may be used in the present invention is not particularly limited, and may typically be prepared by using an enzyme having allulose-converting activity or a microorganism producing such an enzyme, with fructose or sucrose as a substrate.

The enzyme having allulose-converting activity refers to an enzyme capable of converting a substrate including fructose or sucrose into allulose, and may be, for example, an enzyme derived from at least one strains selected from the group consisting of Corynebacterium spp and Microbacterium spp.

In the present invention, allulose may be administered orally to a human or animal either as is or in the form of food or a pharmaceutical composition.

In the allulose composition, allulose as an active ingredient may be included in an amount of 1 g to 60 g, 5 g to 60 g, 5 g to 50 g, 5 g to 40 g, 5 g to 30 g, 5 g to 25 g, 10 g to 25 g, or 10 g to 15 g per day, based on a 60 kg adult.

The allulose usable in the present invention may be in either liquid or powder form and may be included in the composition of the present invention at various concentrations. The allulose may be used as a single component or as part of a mixed composition comprising other saccharides.

The allulose in liquid or powder form may be present in the composition at a content of 50 (w/w) % or more, 60 (w/w) % or more, 70 (w/w) % or more, or 80 (w/w) % or more, and preferably 85 (w/w) % or more, based on total solids. In addition, the crystalline form of allulose may be present in the composition at a content of 98% or more, based on total solids.

The formulations of a pharmaceutical, quasi-drug, or supplement containing allulose is not particularly limited and may be appropriately selected depending on the route of administration. For example, in the case of oral administration, the formulations may be a solid or liquid formulation such as a powder, tablet, sugar-coated tablet, capsule, granule, dry syrup, solution, syrup, lozenge, or drink formulation.

Allulose may be added during the general manufacturing processes of various foods, food additives, and animal feeds. Allulose has a relative sweetness of approximately 70%, and allulose has a quality of taste, physical properties, and processability similar to those of sucrose. Therefore, it can be used in the manufacture of various food products by partially or entirely replacing sucrose, and may be treated similarly to sucrose for use in foods, beverages, food additives, pharmaceuticals, or feeds.

Specific embodiments of the composition according to the present invention may include, for example, beverages, dairy products, edible granules, pastes, seasonings, retort foods, baby foods, fermented foods, preserved foods, processed marine products, processed meat products, processed grain products, food additives, health foods, and animal feeds.

### Effect of the Invention

The present invention can improve intestinal immunity in humans or animals in a simple and effective manner by improving gut leakage in humans or animals, without significant concern on side effects or safety.

### BRIEF DESCRIPTION OF THE DRAWINGS

FIG. 1, FIG. 2a, and FIG. 2b are drawings to show that the mRNA and protein expression levels of factors related to TLR4/NF-KB signaling were reduced by allulose treatment in Caco-2 cells with LPS-induced inflammation according to Example 1.

FIG. 3, FIG. 4a, FIG. 4b, and FIG. 5 are drawings to show that the mRNA and protein expression levels of factors related to TLR4/NF-κB signaling were reduced by allulose treatment. in HT-29 cells with LPS-induced inflammation according to Example 2.

FIG. 6 is a drawing to show that the mRNA and protein expression levels of prostaglandin E2 and COX-2 were reduced by allulose treatment in Caco-2 cells with LPS-induced inflammation according to Example 3.

FIG. 7 is a graph showing the RT-PCR results of mucin expression related to intestinal barrier protection, and the decreased expression levels of MUC2, MUC5AC, and MUC5B in HT-29 cells with LPS-induced inflammation according to Example 3.

FIG. 8 is a graph showing that the TEER values decreased by IFN-γ/TNF-α were increased by allulose treatment in polarized Caco-2 cells according to Example 4.

FIG. 9 is a graph showing that the fluorescence intensity increased by IFN-γ/TNF-α were increased by allulose treatment in polarized Caco-2 cells using FITC-dextran (4 kD) according to Example 5.

FIG. 10 is a graph showing that accumulated ROS was reduced by allulose in Caco-2 cells with LPS-induced inflammation using a DCFH-DA fluorescent probe according to Example 7, indicating reduced intestinal permeability.

FIG. 11 is a graph showing that, the mRNA and protein expression levels of tight junction proteins of ZO-1 and occluding which were reduced by inflammation, were increased by allulose treatment in Caco-2 cells with LPS-induced inflammation according to Example 8.

FIG. 12 shows the results of MTT assay performed after treating human intestinal epithelial cells (HT-29 and Caco-2) with various concentrations of allulose and culturing them for 24 hours according to Example 9.

### MODE FOR INVENTION

The present invention will be described in more detail with reference to the following examples, but the scope of the present invention is not intended to be limited to the following examples.

### Example 1: Evaluation of the Inhibitory Effect of Allulose on the Expression of Factors Related to TLR4/NF-κB Signaling

The anti-inflammatory effect of allulose was confirmed in Caco-2 cells with inflammation induced with LPS (Lipopolysaccharide). The gene and protein expression levels of factors (p50, p65, TLR4, MyD88, IKKα, IKKβ, IKβα, IRAK4, TRAF6) related to TLR4/NF-κB signaling, which is a signaling that mediates the inflammatory response, were confirmed through RT-PCR and Western blot.

Specifically, Caco-2 cells were cultured and differentiated at a density of 2×10⁵ cells/well in 24-well plates and simultaneously treated with allulose dissolved in MEM medium as culture medium of Caco-2 cells, at concentrations of 5, 10, and 20 mM. After 6 hours, LPS (1 μg/ml) was treated to induce inflammation for 24 hours. The culture was performed in an incubator at 5% CO₂ and 37° C.

As a negative control, Caco-2 cells not treated with LPS were used. As a positive control, the differentiated Caco-2 cells which were treated with LPS to induce inflammation but not treated with allulose were used.

After extracting RNA from the cells using Trizol, cDNA was synthesized using a cDNA synthesis kit, and the expression level of the genes was confirmed by qRT-PCR using primers designed for each gene. The PCR conditions used were denaturation at 95° C. for 15 seconds, annealing at 62° C. for 30 seconds, and an elongation process at 72° C. for 1 minute. For protein expression analysis, proteins were extracted from the cultured cells using RIPA lysis buffer. The extracted proteins were pretreated by reaction at 95° C. for 5 minutes, and then separated by electrophoresis on SDS-PAGE. After the proteins were transferred to a PVDF membrane, they were blocked in a 5% (w/v) BSA solution for 1 hour, and the primary antibodies for each factor were diluted 1:1000 in a 5% (w/v) BSA solution and reacted at 4° C. for one day. The membrane reacted with the primary antibody was reacted with the secondary antibody in a tris-buffered saline solution containing 0.1% (v/v) Tween 20 for 1 hour at room temperature. The bands were detected using a LAS 4000 system, and the expression density was calculated using image detection software (Image J software) to calculate the expression amount.

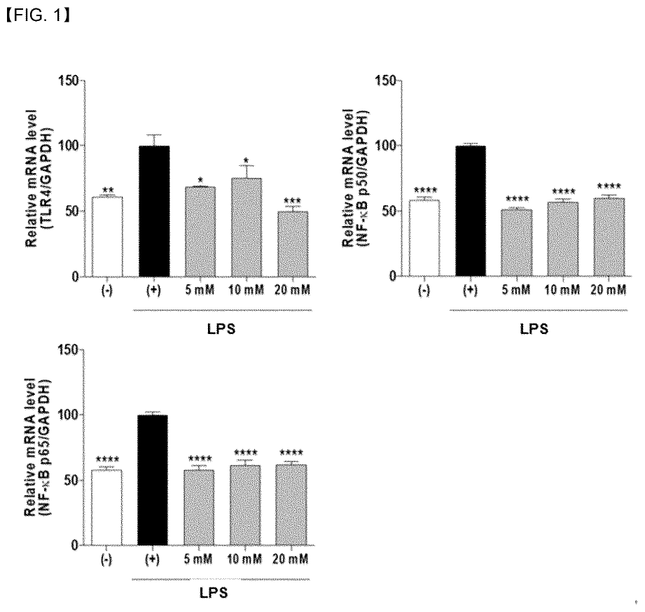

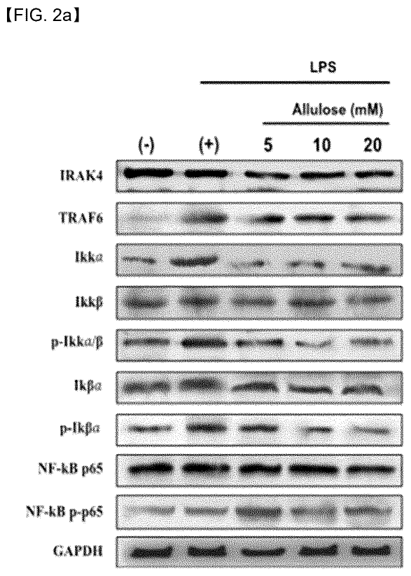

According to the experimental results, a graph was obtained showing that allulose decreased the expression level of factors related to TLR4/NF-κB signaling, which is a signal mediating inflammatory response, in Caco-2 induced with inflammation by LPS. Specifically, the results of analyzing the mRNA expression level by RT-PCR are shown in FIG. 1, and the results of analyzing the protein expression level by Western blotting are shown in FIGS. 2a and 2b. In FIGS. 1, 2a and 2b, the control group (−) represents the expression level of factors related to the TLR4/NF-κB signaling in Caco-2 cells not treated with LPS, and the control group (+) represents an experimental group that used Caco-2 cells treated with LPS to induce inflammation in the same way as the experimental group, but did not being treated with allulose.

From the experimental results, when an inflammatory response occurred by LPS treatment, the expression of TLR4 and NF-κB p65 genes increased, but when being treated with allulose, the expression amount of TLR4 activated by LPS decreased, and thus, the expression amount of NF-κB p65/p50 activated subsequently also decreased. The anti-inflammatory preventive effect was confirmed by suppressing factors related to TLR4/NF-κB activity by allulose treatment in intestinal epithelial cells with induced inflammation.

### Example 2: Evaluation of the Inhibitory Effect of Allulose on the Expression of Factors Related to TLR4/NF-κB Signaling Using HT-29 Cells

HT-29 cells were treated simultaneously with LPS (1 μg/ml) and allulose, and reacted for 8 hours. Specifically, HT-29 cells were seeded at a concentration of 2×10⁵ cells/well in 24-well plates and cultured in an incubator at 37° C. and % CO₂ for 6-7 days. Allulose dissolved in RPMI 1640 medium, which is a HT-29 culture medium, was treated simultaneously at concentrations of 5, 10, and 20 mM, and after 8 hours, LPS (1 μg/ml) was treated to induce inflammation for 24 hours.

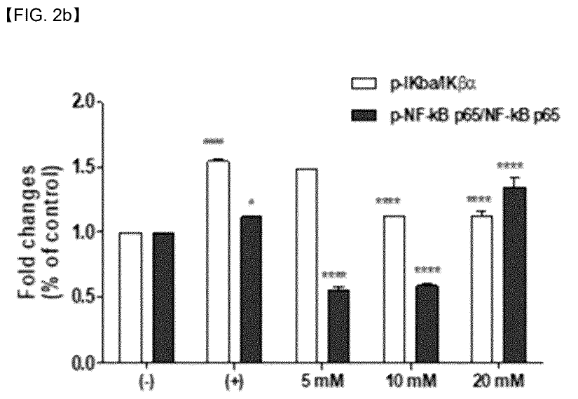

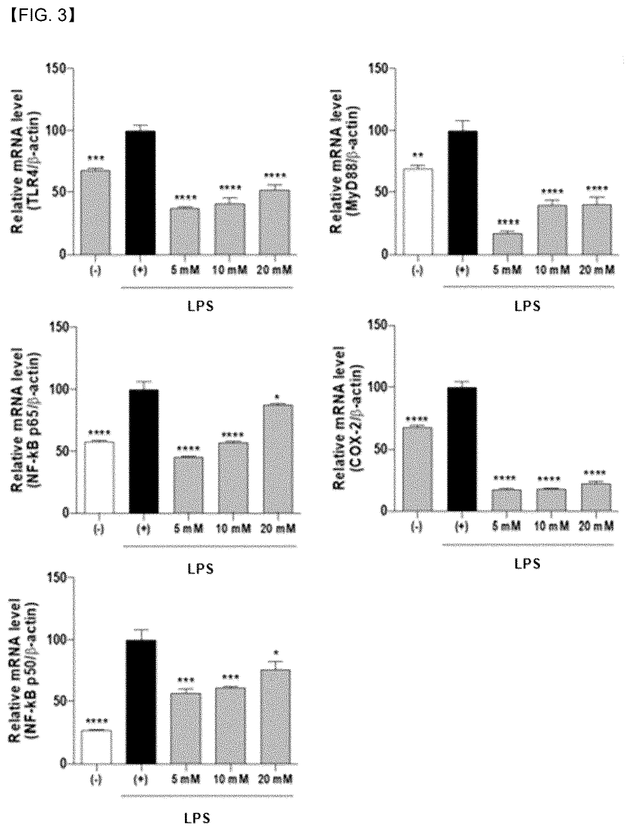

The gene and protein expression levels of Factors involved in TLR4/NF-κB signaling and TLR4/MAPK signaling were confirmed using RT-PCR and Western blot. After extracting RNA from cells using Trizol, cDNA was synthesized using a cDNA synthesis kit, and the gene expression levels were measured by qRT-PCR using primers designed for each gene. The PCR conditions used were denaturation at 95° C. for 15 seconds, annealing at 62° C. for 30 seconds, and elongation at 72° C. for 1 minute. For protein expression analysis, proteins were extracted from cultured cells using RIPA lysis buffer. The extracted proteins were pretreated by reacting at 95° C. for 5 minutes, and then separated by electrophoresis in SDS-PAGE. After transferring the protein to the PVDF membrane, it was blocked in 5% (w/v) BSA solution for 1 hour, and the primary antibody for each factor was diluted 1:1000 with 5% (w/v) BSA solution and reacted at 4° C. for 1 day. The membrane reacted with the primary antibody was reacted with the secondary antibody in tris-buffered saline solution containing 0.1% (v/v) Tween 20 for 1 hour at room temperature. The bands were detected using the LAS 4000 system, and the expression density was calculated using image detection software (image J software) to calculate the expression amount. The results of the experiment are shown in FIGS. 3, 4a, 4b, and 5.

According to the results of the experiment, the graphs showing that allulose decreased the expression level of factors related to TLR4/NF-κB signaling and TLR4/MAPK signaling, which are signals mediating inflammatory response, in HT-29 with LPS-induced inflammation are shown in FIGS. 3 to 5. In FIG. 3, the control group (−) represents the expression level of factors related to TLR4/NF-κB signaling in Caco-2 cells not treated with LPS, and the control group (+) represents the experimental group that used Caco-2 cells treated with LPS to induce inflammation in the same manner as the experimental group, but not treated with allulose.

As a result of the experimental results, allulose decreased the expression level of TLR4 as an inflammatory response-mediated initiating signal, in HT-29 cells with LPS-induced inflammation. Accordingly, the expression level of COX-2 also decreased as the subsequently activated MyD88, IRAK4, TRAF6, NF-κB signal (p50, p65, p-p65, Ikkα, Ikkβ, p-Ikkαβ, Ikβα, p-Ikβα) and MAPK signal (p38, p-p38, JNK, p-JNK, c-Jun, p-c-Jun, c-Fos, p-c-Fos) decreased. This confirmed anti-inflammatory effect by showing that the expression levels of factors related to TLR4/NF-κB signaling and TLR4/MAPK signaling decreased through RT-PCR and Western blot.

### Example 3: Modulation of Intestinal Inflammatory Response by Allulose

Caco-2 cells were cultured in 24-well plates at a density of 2×10⁵ cells/well. The medium was replaced every two days, and allulose was simultaneously treated at concentrations of 5 mM, 10 mM, and 20 mM, and cultured for 6 hours after treatment and reacted for 12 hours after LPS (1 μg/ml) treatment. The supernatant was used for ELISA analysis, and RNA was extracted from the cells using Trizol, and cDNA was synthesized using a cDNA synthesis kit. Then, the expression level of the genes was assessed by qRT-PCR using primers designed for each gene. The PCR conditions used were denaturation at 95° C. for 15 seconds, annealing at 62° C. for 30 seconds, and an elongation process at 72° C. for 1 minute.

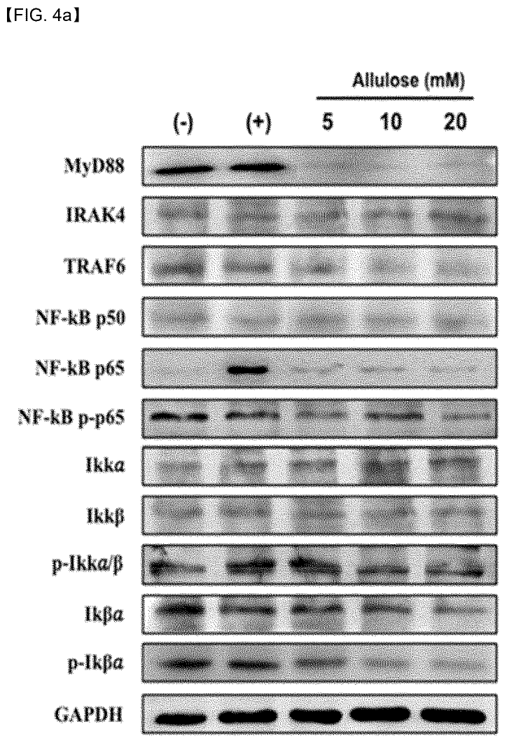

FIG. 6 is a graph showing the inflammation regulating effect by confirming the expression levels of Prostaglandin E2 as a pro-inflammatory mediator, and COX-2 as a key indicator of inflammatory bowel disease, in Caco-2 cells with LPS-induced inflammation LPS using ELISA and RT-PCR according to Example 3, and showing a decrease in the concentration of Prostaglandin E2 and the expression level of COX-2. In FIG. 6, the control group (−) represents the expression level of factors related to the TLR4/NF-κB signaling in HT-29 cells not treated with LPS, and the control group (+) represents an experimental group in which HT-29 cells were treated with LPS to induce inflammation in the same manner as the experimental group, but not treated with allulose.

As a result of the experiment, the expression levels of Prostaglandin E2 as a pro-inflammatory mediator, and COX-2 as a key indicator of inflammatory bowel disease, were confirmed by ELISA and RT-PCR in Caco-2 cells with LPS-induced inflammation. The concentration of Prostaglandin E2 and the expression level of COX-2 were decreased, indicating the effect of regulating inflammation.

### Example 4: Evaluation of Intestinal Mucosal Protective Effect of Allulose Using HT-29 Cells

The effect of allulose on expression level of mucins was confirmed in HT-29 cells with LPS-induced inflammation. Specifically, HT-29 cells were seeded at a concentration of 2×10⁵ cells/well in 24-well plates and cultured in an incubator at 37° C. and % CO₂ for 6-7 days. Allulose dissolved in RPMI 1640 medium of a HT-29 culture medium, was simultaneously treated at concentrations of 5, 10, and 20 mM, and after 8 hours, LPS (1 μg/ml) was treated to induce inflammation for 24 hours. Afterwards, the cells were washed with PBS, RNA was extracted from the cells using Trizol, and cDNA was synthesized using a cDNA synthesis kit. Then, the expression level of the gene was assessed by qRT-PCR using primers produced for mucin genes (MUC2, MUC5AC, MUC5B) that protected the intestinal mucosa. The PCR conditions used were denaturation at 95° C. for 15 seconds, annealing at 62° C. for 30 seconds, and elongation at 72° C. for 1 minute.

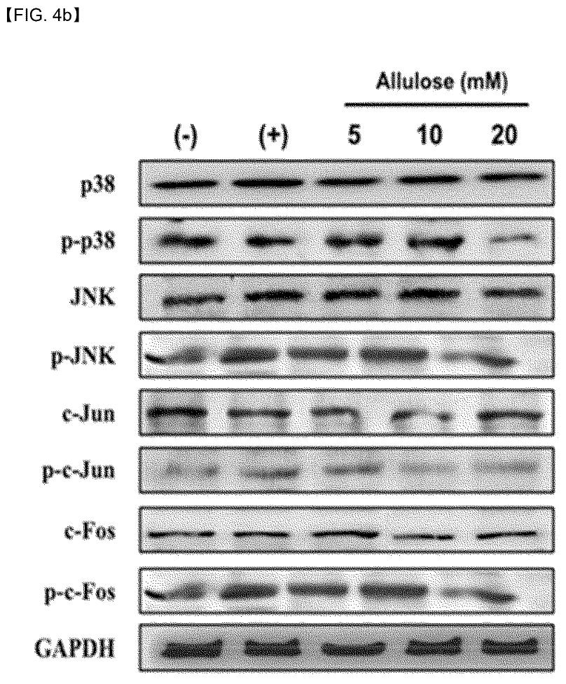

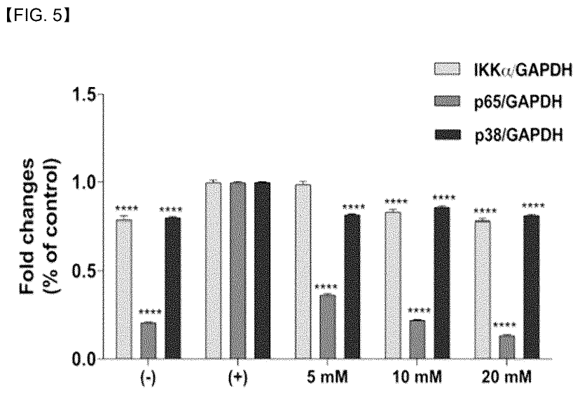

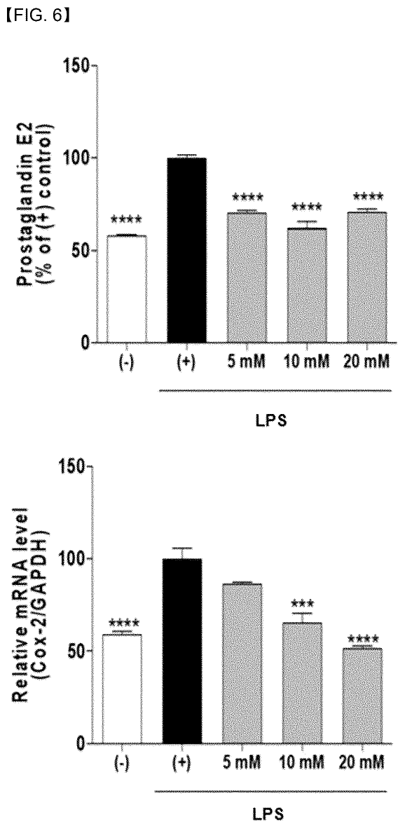

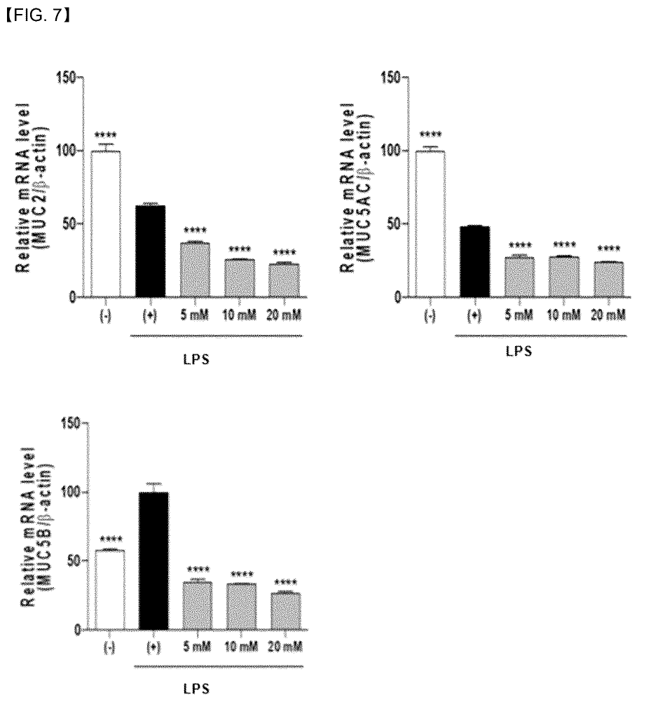

According to the experimental results, the expression level of mucin related to barrier protection in HT-29 cells with LPS-induced inflammation LPS was assessed by RT-PCR. A graph showing that the expression levels of MUC2, MUC5AC, and MUC5B were reduced was shown in FIG. 7. In FIG. 7, the control group (−) represents the expression level of factors related to the TLR4/NF-κB signaling in HT-29 cells not treated with LPS, and the control group (+) represents an experimental group that used HT-29 cells treated with LPS to induce inflammation in the same manner as the above experimental group, but not treated with allulose.

As a result of the experiment, the expression level of mucins related to barrier protection in HT-29 cells with LPS-induced inflammation was assessed by RT-PCR, and the expression levels of MUC2, MUC5AC, and MUC5B were decreased, confirming that allulose protected the area around the intestine.

### Example 5: Evaluation of Improvement in Intestinal Permeability by Allulose (Measurement of Transepithelial Electrical Resistance in Intestinal Cells)

Transepithelial electrical resistance (TEER) was measured as an indicator of the adhesive strength between intestinal epithelial cells using allulose as an active ingredient, and the electrical resistance values of the upper and lower layers of intestinal cells for allulose materials were examined.

Specifically, Caco-2 cells were seeded in the upper layer of a Transwell insert at a concentration of 5×10⁴ cells/well, and 600 μl of medium without cells was added to the lower layer and cultured. The medium was replaced every 2 days, and cultured for 3 weeks to form polarization. After polarization formation, the upper layer with cells was pre-treated with IFN-γ (10 ng/ml) for 24 hours, allulose was treated at various concentrations for 1 hour, and TNF-α (10 ng/ml) was treated for 6 hours. After the reaction was completed, the electrical resistance value TEER was measured using a Millicell ERS-2 Voltohmmeter.

According to the experimental results, the graph showing the values of the electrical resistance value (TEER) decreased by IFN-γ/TNF-α in polarized Caco-2 cells and the values of the electrical resistance value (TEER) increased by allulose was shown in FIG. 8. The values of the electrical resistance value (TEER) decreased by IFN-γ/TNF-α in polarized Caco-2 cells and the values of the electrical resistance value (TEER) increased by allulose were confirmed.

### Example 6: Evaluation of Transepithelial Permeability of Intestinal Cells by Allulose

When FITC-dextran (4 kD) as a fluorescently labeled protein, is applied to cells with barrier damage caused by inflammatory factors, it refers to increased permeability. To test the effect of allulose as an active ingredient on material permeability of barrier, paracellular permeability was confirmed using FITC-dextran.

Specifically, Caco-2 cells were seeded in the upper layer of the transwell insert at a concentration of 5×10⁴ cells/well, and 600 μL of medium without cells was added to the lower layer and cultured. The medium was replaced every 2 days, and cultured for 3 weeks to form polarization. After polarization formation, the upper layer with cells was pre-treated with IFN-γ (10 ng/ml) for 24 hours, allulose was treated at concentrations of 5, 10, and 20 mM for 1 hour, and TNF-α (10 ng/ml) was treated for 6 hours. FITC-dextran (100 μg/ml) diluted in medium was treated to the transwell and reacted for 4 hours. Paracellular permeability was measured at excitation 488 nm and emission 535 nm using fluorescence absorbance.

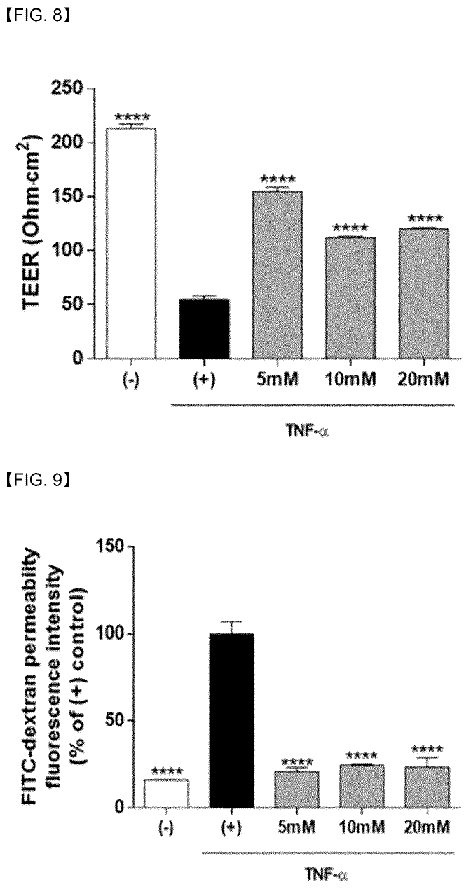

According to the experimental results, by using through FITC-dextran (4 kD) in polarized Caco-2 cells, the graph showing the increased fluorescence intensity by IFN-γ/TNF-α was increased by allulose was in FIG. 9.

### Example 7: Evaluation of ROS Scavenging Activity of Allulose

Accumulated ROS was reduced by allulose in Caco-2 cells with LPS-induced inflammation using a DCFH-DA fluorescent probe, indicating decreased intestinal permeability.

Caco- 2 cells were seeded at 5×10⁴ cells/well in 96-well plates and cultured. After simultaneous treatment with LPS (1 μg/ml) and allulose at concentrations of 5 mM, 10 mM, and 20 mM, the cells were cultured for 12 h under conditions of 5% CO₂ and 37° C. After removing the culture supernatant, DCF-DA was added to the medium at a concentration of 20 μM and cultured in an incubator for 30 min. After culture, the cells were washed with PBS and the amount of ROS production was measured using fluorescence absorbance at excitation of 488 nm and emission of 535 nm.

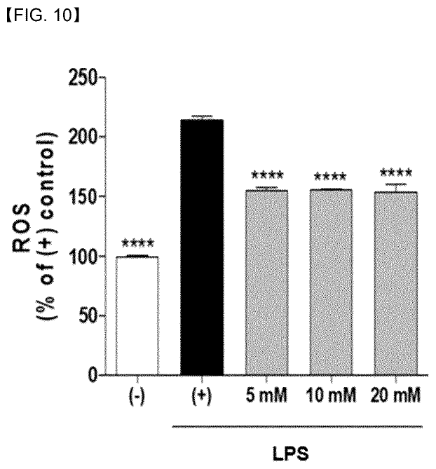

FIG. 10 is a graph showing that accumulated ROS is reduced by allulose in Caco-2 cells with LPS-induced inflammation according to Example 7, indicating that intestinal permeability was reduced.

### Example 8: Increased Expression of Tight Junction Factors by Allulose

Caco-2 cells were cultured in 24-well plates at a density of 2×10⁵ cells/well. The medium was replaced every other day, and the cells were cultured for 6 hours after treatment with 5, 10, and 20 mM allulose, and reacted for 12 hours after treatment with LPS (1 μg/ml). After extracting RNA from the cells using Trizol, cDNA was synthesized using a cDNA synthesis kit, and the gene expression level was confirmed by qRT-PCR using primers designed for each gene. The PCR conditions used were denaturation at 95° C. for 15 seconds, annealing at 62° C. for 30 seconds, and elongation at 72° C. for 1 minute. For protein expression analysis, proteins were extracted from the cultured cells using RIPA lysis buffer. The extracted proteins were pretreated by reacting at 95° C. for 5 minutes, and then separated by electrophoresis on SDS-PAGE. After transferring the proteins to the PVDF membrane, they were blocked in a 5% (w/v) BSA solution for 1 hour, and the primary antibodies for each factor were diluted 1:1000 in a 5% (w/v) BSA solution and reacted at 4° C. for 1 day. The membrane reacted with the primary antibodies was reacted with the secondary antibodies in a tris-buffered saline solution containing 0.1% (v/v) Tween 20 for 1 hour at room temperature. The bands were detected using a LAS 4000 system, and the expression density was calculated using image detection software (image J software) to calculate the expression amount.

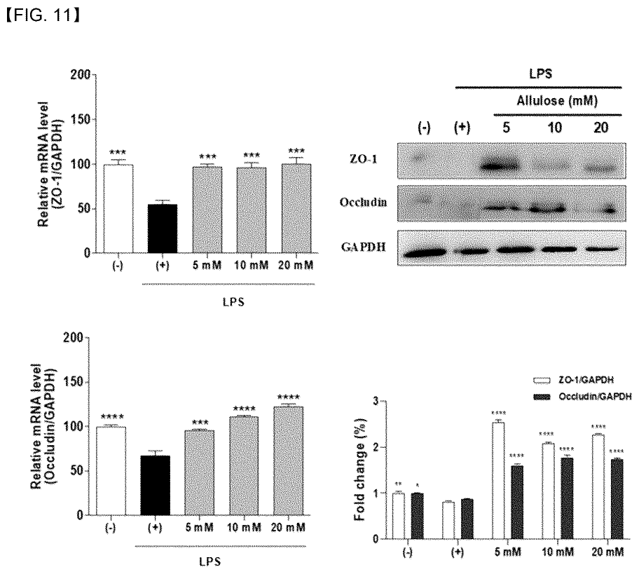

According to the experiment, the results showing that the mRNA and protein expression levels of ZO-1 and Occludin, which were tight junction factors decreased by inflammation, were increased in Caco-2 cells with LPS-induced inflammation as shown in FIG. 11. The mRNA and protein expression levels of ZO-1 and Occludin, which were tight junction factors decreased by inflammation were increased by treatment of allulose in Caco-2 cells with LPS-induced inflammation. This shows that intestinal permeability, i.e. tight junction, damaged by induced inflammation was improved by allulose.

### Example 9: Toxicity Evaluation of Allulose

Human intestinal epithelial cells of HT-29 and Caco-2, were treated with allulose at concentrations of 5, 10, and 20 mM, respectively, cultured for 24 hours, and then were performed for MTT assay.

Specifically, HT-29 and Caco-2 cells were seeded at a density of 5×10⁴ cells/well in a 96-well plate and cultured under conditions of 5% CO₂ and 37° C. The medium was removed, added with 5 mg/ml MTT solution, and reacted for 2 hours. 200 μL/well of dimethyl sulfoxide (DMSO) was added, and formazan was dissolved at room temperature for 10 minutes. The absorbance was measured at 590 nm to calculate the cell activity (%). In addition, the cytotoxicity was measured by comparing the cell activity with and without the sample.

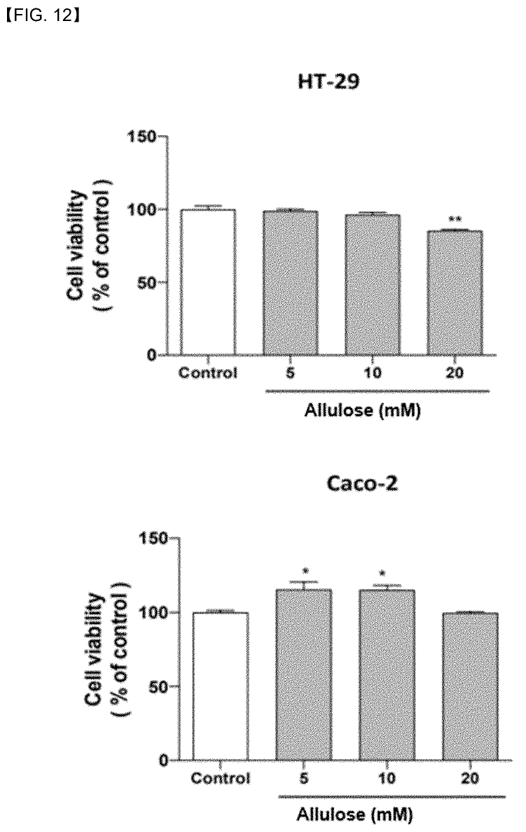

The results obtained from the experiments are shown in the cytotoxicity evaluation for allulose, and specifically, the toxicity evaluation according to the allulose concentration is shown in FIG. 12. FIG. 12 shows the results of the toxicity evaluation for HT-29 & Caco-2.

According to the toxicity evaluation results, it was confirmed that allulose was not toxic at concentrations of 5 mM, 10 mM, and 20 mM. These results confirmed that allulose effectively protected barrier cells.

1. A composition for preventing, improving, or treating a leaky gut or a leaky gut syndrome, comprising allulose as an active ingredient.

2. The composition according to claim 1, wherein the leaky gut is caused by an increased permeability of intestinal mucosa, a damage to intestinal mucosa, or an intestinal inflammation.

3. The composition according to claim 1, wherein the leaky gut has characteristics of a decreased expression of TLR4, an increased expression of Prostaglandin E2 and COX-2, a decreased adhesion strength between intestinal epithelial cells, or an increased expression of ZO-1 and Occludin.

4. The composition according to claim 1, wherein the composition has at least activities of allulose selected from the group consisting of an enhancing activity of intestinal mucosal immunity, an anti-inflammatory activity, a protective activity of intestinal mucosa, an improving activity of intestinal permeability, or a scavenging activity of reactive oxygen species (ROS).

5. The composition of claim 4, wherein the enhancing activity of intestinal mucosal immunity is regulation of the expression of factors related to TLR4/NF-κB signaling and TLR4/MAPK signaling involved in inflammatory reaction regulation, or preferably at least a factor related to TLR4/NF-κB signaling selected from the group consisting of p50, p65, TNF-α, IL-6, IL-8, COX-2, TLR4, MyD88, IKKα, IKKβ, IκBα, IRAK4, and TRAF6, or at least a factor related to MAPK signaling selected from the group consisting of p38, p-p38, JNK, p-JNK, c-Jun, p-c-Jun, c-Fos, and p-c-Fos;
wherein the anti-inflammatory activity is reduction or inhibition of the expression of at least one selected from the group consisting of prostaglandin E2 as a pro-inflammatory mediator, and COX-2 as a key indicator of inflammatory bowel disease;
wherein the protective activity of intestinal mucosa is reduction or inhibition of the expression of at least one selected from the group consisting of MUC2, MUC5AC, and MUC5B, as a mucin protecting intestinal mucosa;
wherein the improving activity of intestinal permeability is improvement of increased intestinal permeability induced by barrier damage which is caused by inflammatory factors, improvement of adhesion strength between intestinal epithelial cells, or increased expression of ZO-1 and Occludin as tight junction factors; or
wherein the scavenging activity of ROS is increase in the expression of at least one selected from the group consisting of PI3K, AKT, Nrf2, and HO-1, as factors related to Nrf2/HO-1 signaling activated by ROS.

6. The composition of claim 1, wherein the allulose is a TLR4 inhibitor or a TLR4 antagonist.

7. The composition of claim 6, wherein the allulose, as a TLR4 inhibitor, regulates expression of at least one selected from the group consisting of p50, p65, TNF-α, IL-6, IL-8, COX-2, TLR4, MyD88, IKKα, IKKβ, IκBα, IRAK4, and TRAF6, as factors related to TLR4/NF-κB signaling.

8. The composition of claim 1, wherein the preventing, improving, or treating leaky gut or leaky gut syndrome is not caused by a prebiotic property of allulose in regulating a growth of intestinal microbiota.

9. The composition of claim 1, wherein the allulose is included in an amount of 1 g to 60 g per day based on a 60 kg adult.

10. The composition of claim 1, wherein the allulose is provided as a saccharide syrup containing allulose.

11. The composition of claim 1, wherein the allulose is provided as a liquid form or powdery form.

12. The composition of claim 11, wherein the powdery form is amorphous or crystalline.

13. A food composition for preventing, improving, or treating a leaky gut or a leaky gut syndrome, comprising allulose as an active ingredient.

14. The food composition according to claim 13, wherein the leaky gut is caused by an increased permeability of intestinal mucosa, a damage to intestinal mucosa, or an intestinal inflammation.

15. The food composition according to claim 13, wherein the leaky gut has characteristics of a decreased expression of TLR4, an increased expression of Prostaglandin E2 and COX-2, a decreased adhesion strength between intestinal epithelial cells, or an increased expression of ZO-1 and Occludin.

16. The food composition according to claim 13, wherein the allulose is a TLR4 inhibitor or a TLR4 antagonist.

17. The food composition according to claim 13, wherein the allulose is included in an amount of 1 g to 60 g per day based on a 60 kg adult.

18. A method of preventing, improving, or treating a leaky gut or a leaky gut syndrome, comprising administering allulose as an active ingredient to a subject in need thereof.
